(19) United States
(12) Patent Application Publication
(10) Pub. No.: US 2021/0046465 A1
(43) Pub. Date: Feb. 18, 2021

(54) METHODS OF CONTROLLING SURFACE TENSION

(71) Applicant: LyondellBasell Acetyls, LLC, Houston, TX (US)

(72) Inventors: David L. Ramage, Friendswood, TX (US); Rene Zamarripa, Houston, TX (US)

(73) Assignee: LyondellBasell Acetyls, LLC, Houston, TX (US)

(21) Appl. No.: 16/990,639

(22) Filed: Aug. 11, 2020

Related U.S. Application Data
(60) Provisional application No. 62/885,639, filed on Aug. 12, 2019.

(51) Int. Cl. B01J 31/02; B01J 23/46; B01J 31/20; B01J 31/04; C07C 53/08

(52) U.S. Cl. CPC B01J 31/0267; B01J 23/464; B01J 31/20; B01J 2231/40; C07C 53/08; B01J 2531/822; B01J 31/04

(57) ABSTRACT

Reactor liquids, compositions, and methods of forming acetic acid, which may reduce catalyst loss. The reactor liquids and compositions may include, and the methods may use, a tri-aliphatic hydrocarbyl phosphine oxide. The carbonylation catalyst used in the methods may include rhodium. A composition comprising: acetic acid; water; and at least one tri-aliphatic hydrocarbyl phosphine oxide; wherein the acetic acid is present in the composition at an amount of about 60% to about 80%, by weight, based on the weight of the composition; wherein the water is present in the composition at an amount of about 0.1% to about 6%, by weight, based on the weight of the composition; and wherein the at least one tri-aliphatic hydrocarbyl phosphine oxide is present in the composition at an amount of about 2% to about 20%, by weight, based on the weight of the composition.

### PRIOR RELATED APPLICATIONS

This application claims the benefit of priority to U.S. Provisional Application No. 62/885,639, filed Aug. 12, 2019, which is incorporated here by reference in its entirety.

### BACKGROUND

A mechanism of catalyst loss in various processes can include reactor liquid drop entrainment. For example, in processes for producing glacial acetic acid (GAA), a mechanism that may cause the loss of a catalyst, such as rhodium, can include reactor liquid droplet entrainment in a vapor stream, e.g., a vapor stream transported from a flash tank to a purification section of a plant.

Since the year 2000, rhodium prices have varied between $600-$10,000 per troy ounce; and has averaged from $1000 to $2000 per troy ounce. Therefore, if the rhodium usage rate at a facility is about 120 troy ounces per month, and the price of rhodium is $1000 per troy ounce, then the facility would spend about $1.5 MM on rhodium per year.

There remains a need for processes and/or additives that reduce catalyst loss, including processes and/or additives that increase surface tension, thereby increasing average droplet size and reducing entrainment in a vapor stream.

### BRIEF SUMMARY

Provided herein are reactor liquids and compositions that include at least one tri-aliphatic hydrocarbyl phosphine oxide, and methods for the production of acetic acid in the presence of at least one tri-aliphatic hydrocarbyl phosphine oxide. It has been surprisingly discovered that at least one tri-aliphatic hydrocarbyl phosphine oxide can increase surface tension of the reactor liquids and compositions described herein, including those that include no more than 6%, by weight, of water (e.g., 4%, by weight, or less of water). The increase in surface tension may eliminate or reduce catalyst loss during processes, such as processes for producing acetic acid, e.g., glacial acetic acid.

In one aspect, composition(s) are provided. Such compositions may be used in conjunction with a reactor and therefore be referred to as reactor liquid. In some embodiments, the composition or reactor liquid include acetic acid, water, and at least one tri-aliphatic hydrocarbyl phosphine oxide. In some embodiments, the acetic acid is present in the composition or reactor liquid at an amount of about 60% to about 80%, by weight, based on the weight of the composition or reactor liquid. In some embodiments, water is present in the composition or reactor liquid at an amount of about 0.1% to about 6%, by weight, based on the weight of the composition or reactor liquid. In some embodiments, at least one tri-aliphatic hydrocarbyl phosphine oxide is present in the composition or reactor liquid at an amount of about 2% to about 10%, by weight, based on the weight of the composition or reactor liquid.

In another aspect, methods for the production of acetic acid are provided. In some embodiments, the methods include contacting (i) methanol, (ii) carbon monoxide, (iii) water, (iv) a carbonylation catalyst, and (iv) at least one tri-aliphatic hydrocarbyl phosphine oxide in a carbonylation reactor to produce a reactor liquid, wherein the reactor liquid includes the acetic acid.

Additional aspects will be set forth in part in the description which follows, and in part will be obvious from the description, or may be learned by practice of the aspects described herein. The advantages described herein may be realized and attained by means of the elements and combinations particularly pointed out in the appended claims. It is to be understood that both the foregoing general description and the following detailed description are exemplary and explanatory only and are not restrictive.

### BRIEF DESCRIPTION OF THE DRAWINGS

FIG. 1 depicts a Pareto chart, which demonstrates the significance of the data of Example 1.

FIG. 2 depicts a plot of predicted surface tensions of various liquids, including liquids that include an embodiment of a tri-aliphatic hydrocarbyl phosphine oxide.

### DETAILED DESCRIPTION

Provided herein are compositions, reactor liquids and methods for the production of acetic acid. The composition(s) or reactor liquids may be liquids that are produced as part of a method for producing acetic acid. The methods may include methods for the production of glacial acetic acid, which is encompassed by the term “acetic acid” as used herein. Glacial acetic acid refers to acetic acid that is undiluted, meaning that the acetic acid has a water concentration of about 0.15 wt % or less, based on the total weight of acetic acid and water.

### Composition(s)/Reactor Liquid(s)

Provided herein are compositions or reactor liquids that may include acetic acid, water, and at least one tri-aliphatic hydrocarbyl phosphine oxide. The composition(s) or reactor liquids may include one or more other components, such as an additive, as described herein.

In some embodiments, the acetic acid is present in the composition or reactor liquid at an amount of about 50% to about 80%, by weight, based on the weight of the composition or reactor liquid. In some embodiments, the acetic acid is present in the composition or reactor liquid at an amount of about 55% to about 75%, by weight, based on the weight of the composition or reactor liquid. In some embodiments, the acetic acid is present in the composition or reactor liquid at an amount of about 70%, by weight, based on the weight of the composition or reactor liquid.

In some embodiments, the water is present in the composition or reactor liquid at an amount of about 0.1% to about 10%, by weight, based on the weight of the composition or reactor liquid. In some embodiments, the water is present in the composition or reactor liquid at an amount of about 0.1% to about 6%, by weight, based on the weight of the composition or reactor liquid. In some embodiments, the water is present in the composition or reactor liquid at an amount of about 0.1% to about 5%, by weight, based on the weight of the composition or reactor liquid. In some embodiments, the water is present in the composition or reactor liquid at an amount of about 0.1% to about 4%, by weight, based on the weight of the composition or reactor liquid. In some embodiments, the water is present in the composition or reactor liquid at an amount of about 0.1% to about 3%, by weight, based on the weight of the composition or reactor liquid. In some embodiments, the water is present in the composition or reactor liquid at an amount of about 0.1% to about 2%, by weight, based on the weight of the composition or reactor liquid.

In some embodiments, the at least one tri-aliphatic hydrocarbyl phosphine oxide is present in the composition or reactor liquid at an amount of about 2% to about 20%, by weight, based on the weight of the composition or reactor liquid. In some embodiments, the at least one tri-aliphatic hydrocarbyl phosphine oxide is present in the composition or reactor liquid at an amount of about 2% to about 15%, by weight, based on the weight of the composition or reactor liquid. In some embodiments, the at least one tri-aliphatic hydrocarbyl phosphine oxide is present in the composition or reactor liquid at an amount of about 2% to about 6%, by weight, based on the weight of the composition or reactor liquid. In some embodiments, the at least one tri-aliphatic hydrocarbyl phosphine oxide is present in the composition or reactor liquid at an amount of about 3% to about 10%, by weight, based on the weight of the composition or reactor liquid.

### Tri-Aliphatic Hydrocarbyl Phosphine Oxides

The compositions/reactor liquids provided herein may include at least one tri-aliphatic hydrocarbyl phosphine oxide. As used herein, the phrase “tri-aliphatic hydrocarbyl phosphine oxide” refers to a compound that includes a phosphorus atom that is substituted with one oxo moiety, and three aliphatic hydrocarbyl moieties. As used herein the phrases “aliphatic hydrocarbyl”, “aliphatic hydrocarbyl moieties”, and the like refer to non-aromatic hydrocarbons that may be saturated or unsaturated. In some embodiments, the aliphatic hydrocarbyl moieties are non-aromatic, non-cyclic hydrocarbons that may be saturated or unsaturated.

In some embodiments, the at least one tri-aliphatic hydrocarbyl phosphine oxide includes a compound of formula (I):

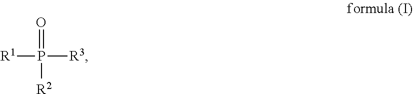

wherein R¹, R², and R³ are independently selected from a monovalent C₁-C₂₀ aliphatic hydrocarbyl.

In some embodiments, R¹, R², and R³ of formula (I) are independently selected from a monovalent C₄-C₁₅ aliphatic hydrocarbyl. In some embodiments, R¹, R², and R³ of formula (I) are independently selected from a monovalent C₆-C₁₃ aliphatic hydrocarbyl. In some embodiments, R¹, R², and R³ of formula (I) are independently selected from a monovalent C₆-C₁₀ aliphatic hydrocarbyl. In some embodiments, R¹, R², and R³ of formula (I) are independently selected from a monovalent C₆-C₈ aliphatic hydrocarbyl.

In some embodiments, R¹, R², and R³ of formula (I) are n-octyl groups.

In some embodiments, R¹, R², and R³ of formula (I) are n-hexyl groups.

In some embodiments, R¹ and R² of formula (I) are n-hexyl groups, and R³ of formula (I) is an n-octyl group.

In some embodiments, R¹ and R² of formula (I) are n-octyl groups, and R³ of formula (I) is an n-hexyl group.

In some embodiments, the at least one tri-aliphatic hydrocarbyl phosphine oxide includes one or more of a compound of formula (Ia), a compound of formula (Ib), a compound of formula (Ic), and a compound of formula (Id). In some embodiments, the at least one tri-aliphatic hydrocarbyl phosphine oxide includes a compound of formula (Ia), a compound of formula (Ib), a compound of formula (Ic), and a compound of formula (Id):

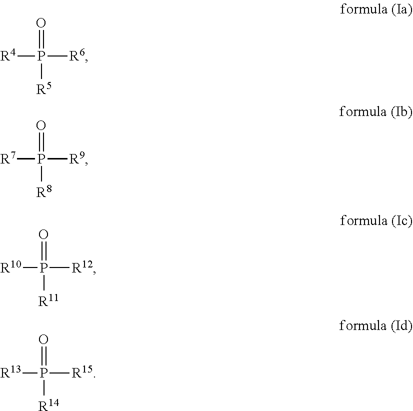

wherein (i) each of R⁴, R⁵, and R⁶ is a monovalent aliphatic C₅-C₇ hydrocarbyl, (ii) each of R⁷, R⁸, and R⁹ is a monovalent aliphatic C₇-C₉ hydrocarbyl, (iii) R¹⁰, R¹¹, and R¹² are independently selected from a monovalent aliphatic C₅-C₉ hydrocarbyl, and (iv) R¹³, R¹⁴, and R¹⁵ are independently selected from a monovalent aliphatic C₅-C₉ hydrocarbyl. In some embodiments, (i) each of R⁴, R⁵, and R⁶ is an n-hexyl group, (ii) each of R⁷, R⁸, and R⁹ is an n-octyl group, (iii) R¹⁰ and R¹¹ are n-hexyl groups, and R¹² is an n-octyl group, and (iv) R¹³ and R¹⁴ are n-octyl groups, and R¹⁵ is an n-hexyl group.

When the at least one tri-aliphatic hydrocarbyl phosphine oxide includes one or more of a compound of formula (Ia), a compound of formula (Ib), a compound of formula (Ic), and a compound of formula (Id), the compound of formula (Ia), the compound of formula (Ib), the compound of formula (Ic), and/or the compound of formula (Id) may be present at any weight ratio or mole ratio to each other or to another component of a composition or reactor liquid. For example, in some embodiments, the at least one tri-aliphatic hydrocarbyl phosphine oxide includes a compound of formula (Ia), a compound of formula (Ib), a compound of formula (Ic), and a compound of formula (Id), and the compound of formula (Ia), the compound of formula (Ib), the compound of formula (Ic), and the compound of formula (Id) are present at a weight ratio of about 1:0.5-1.5:0.5-1.5:0.5-1.5, or about 1:1:1:1.

### Methods for the Production of Acetic Acid

Methods for the production of acetic acid also are provided herein. In some embodiments, the methods include contacting (i) methanol, (ii) carbon monoxide, (iii) water, (iv) a carbonylation catalyst, and (iv) at least one tri-aliphatic hydrocarbyl phosphine oxide in a carbonylation reactor to produce a reactor liquid, wherein the reactor liquid includes the acetic acid. A reactor liquid produced by the methods provided herein may include any of the compositions or reactor liquids provided herein.

In some embodiments, the methods include providing methyl acetate as a co-feed. As an alternative to methyl acetate as a co-feed, the contacting of the methods herein can take place in the presence of a mixture of methyl acetate and methanol from byproduct streams of the hydrolysis/methanolysis of polyvinyl acetate. Additionally, methyl acetate may be generated during a process.

The carbonylation catalysts used in the methods provided herein may include any carbonylation catalysts. In some embodiments, the carbonylation catalysts include rhodium, iridium, or a combination thereof. In some embodiments, the carbonylation catalyst also includes a catalyst stabilizer, a catalyst promoter, or a combination thereof.

When the carbonylation catalyst includes rhodium, the carbonylation catalyst can include rhodium metal, a rhodium compound, or a combination thereof. The rhodium compounds can include rhodium salts, rhodium oxides, rhodium acetates, organo-rhodium compounds, coordination compounds of rhodium, or a combination thereof. Non-limiting examples of rhodium compounds include Rh₂(CO)₄I₂, Rh₂(CO)₄Br₂, Rh₂(CO)₄Cl₂, Rh(CH₃CO₂)₂, Rh(CH₃CO₂)₃, or [H]Rh(CO)₂I₂.

When the carbonylation catalyst includes iridium, the carbonylation catalyst may include iridium metal, an iridium compound, or a combination thereof. The iridium compounds can include iridium salts, iridium oxides, iridium acetates, iridium oxalates, iridium acetoacetates, coordination compounds of iridium, or a combination thereof. Non-limiting examples of iridium compounds include IrCl₃, IrI₃, IrBr₃, [Ir(CO)₂I]₂, [Ir(CO)₂Cl]₂, [Ir(CO)₂Br]₂, [Ir(CO)₄I₂]⁻H⁺, [Ir(CO)₂Br₂]⁻H⁺, [Ir(CO)₂I₂]⁻H⁺, [Ir(CH₃)I₃(CO)₂]⁻H⁺, Ir₄(CO)₁₂, IrCl₃4H₂O, IrBr₃4H₂O, Ir₃(CO)₁₂, Ir₂O₃, IrO₂, Ir(acac)(CO)₂, Ir(acac)₃, Ir(Ac)₃, [Ir₃O(OAc)₆(H₂O)₃][OAc], or H₂[IrCl₆].

The methods provided herein may use, and/or the compositions or reactor liquids provided herein may also include, a catalyst stabilizer. The catalyst stabilizer may include a metal iodide salt, such as lithium iodide, or a non-salt stabilizer, such as a pentavalent Group VA oxide. The at least one tri-aliphatic hydrocarbyl phosphine oxide may act as a catalyst stabilizer.

The methods provided herein may use, and/or the compositions or reactor liquids provided herein may also include a catalyst promoter, such as methyl iodide. Methyl iodide may be added directly to a process or generated by adding hydrogen iodide to a process.

The contacting of the methods provided herein may be performed at any temperature and/or pressure that may form acetic acid. In some embodiments, the contacting of the methods provided herein occurs at a temperature of about 150 degrees Celsius (° C.) to about 250° C. and under a pressure of about 200 psig (1380 kPa) to about 2,000 psig (13,800 kPa).

After a composition or reactor liquid is produced, the composition or reactor liquid may be passed downstream to one or more separation units. Separation unit(s) may include a vessel or step in an acetic acid process which separates a feed stream into two or more separate exit streams where the two exit streams differ from one another in some aspect. The separation can be based on methods such as separation by physical characteristics (e.g., density, volatility, boiling point, phase, absorbance, and adsorbance) or other characteristics used for separating chemical components.

The separation unit(s) can include, but are not limited to, a flash tank, a light ends distillation column, a decanter, a drying column, a heavy ends distillation column, or a combination thereof.

In a flash tank, an acetic acid product stream may be withdrawn from a reactor and separated into (a) a liquid fraction that may include a carbonylation catalyst and a catalyst stabilizer, and (b) a vapor fraction that may include the acetic acid product, reactants, water, methyl iodide, and impurities generated during the carbonylation reaction, including acetaldehyde. The liquid fraction may be recycled to the carbonylation reactor. The vapor fraction may then be passed to a distillation column.

In a light ends distillation column, a vapor fraction may be separated based upon boiling point into at least (a) an overhead fraction including methyl iodide, water, methanol, methyl acetate, acetic acid, alkanes, and acetaldehyde, and (b) an acetic acid stream including acetic acid, water, hydrogen iodide, and heavy impurities, such as propionic acid.

In a decanter, the overhead fraction may be condensed and separated by density to (a) a lighter aqueous phase and (b) a heavier organic phase. The lighter aqueous phase may contain one or more of water, acetic acid, methyl acetate, methyl iodide, acetaldehyde, and alkanes, and have a density of about 1.0 grams per cubic centimeter to about 1.10 grams per cubic centimeter. Like the lighter aqueous phase, the heavier organic phase may contain water, acetic acid, methyl acetate, methyl iodide, acetaldehyde and alkanes; the organic phase may also include higher concentrations of methyl iodide and alkanes. The heavier organic phase may have a density of about 1.4 grams per cubic centimeter to about 2.2 grams per cubic centimeter. The lighter aqueous phase may be recycled to the reactor or to the light ends distillation column.

In a drying column, the residual water may be removed from the acetic acid stream. In a heavy ends distillation column, the heavy impurities may be removed from the acetic acid stream.

In the descriptions provided herein, the terms “includes,” “is,” “containing,” “having,” and “comprises” are used in an open-ended fashion, and thus should be interpreted to mean “including, but not limited to.” When methods or compositions are claimed or described in terms of “comprising” or “including” various elements or features, the methods can also “consist essentially of” or “consist of” the various components or features, unless stated otherwise.

Moreover, it is also to be understood that the lettering of process steps or ingredients is a convenient means for identifying discrete activities or ingredients and the recited lettering can be arranged in any sequence, unless expressly indicated.

The terms “a,” “an,” and “the” are intended to include plural alternatives, e.g., at least one. For instance, the disclosure of “a carbonylation catalyst,” and the like, is meant to encompass one, or mixtures or combinations of more than one carbonylation catalyst, unless otherwise specified.

Various numerical ranges may be disclosed herein. When Applicant discloses or claims a range of any type, Applicant's intent is to disclose or claim individually each possible number that such a range could reasonably encompass, including end points of the range as well as any sub-ranges and combinations of sub-ranges encompassed therein, unless otherwise specified. Moreover, numerical end points of ranges disclosed herein are approximate. As a representative example, Applicant discloses that, in some embodiments, at least one tri-aliphatic hydrocarbyl phosphine oxide is present in a composition or reactor liquid at an amount of about 2% to about 20%, by weight, based on the weight of the composition or reactor liquid. This disclosure should be interpreted as encompassing values of about 2% and about 20%, by weight, and further encompasses “about” each of 3%, 4%, 5%, 6%, 7%, 8%, 9%, 10%, 11%, 12%, 13%, 14%, 15%, 16%, 17%, 18%, and 19%, including any ranges and sub-ranges between any of these values.

The present embodiments are illustrated herein by referring to various embodiments, which are not to be construed in any way as imposing limitations upon the scope thereof. On the contrary, it is to be understood that resort may be had to various other aspects, embodiments, modifications, and equivalents thereof which, after reading the description herein, may suggest themselves to one of ordinary skill in the art without departing from the spirit of the present embodiments or the scope of the appended claims. Thus, other aspects of the embodiments will be apparent to those skilled in the art from consideration of the specification and practice of the embodiments disclosed herein.

The present disclosure is further illustrated by the following examples, which are not to be construed in any way as imposing limitations upon the scope thereof. On the contrary, it is to be understood that resort may be had to various other aspects, embodiments, modifications, and equivalents thereof which, after reading the description herein, may suggest themselves to one of ordinary skill in the art without departing from the spirit of the present disclosure or the scope of the appended claims. Thus, other aspects of this disclosure will be apparent to those skilled in the art from consideration of the specification and practice of the disclosure disclosed herein.

### EXAMPLES

### Example 1—Surface Tension Testing

The experiments of this example were designed to determine the effect of varying the concentrations of various components of a GAA liquid.

The three factor, two level design with center-point resulted in a model with good predictive capability, exemplified by a r² pred. of about 90%. All factors and interactions were found to be significant with increasing concentrations of water, tri-phenyl phosphine oxide (TPPO), and the embodiment of the at least one tri-aliphatic hydrocarbyl phosphine oxide increasing surface tension. The model was used to predict the surface tensions of GAA liquids across a range of concentrations of several components.

A series of GAA liquids were prepared for testing. The GAA liquids of this example included the components of the following table.

TABLE 1
Components of Liquids of this Example

| Component | Weight Percentage |
|---|---|
| Glacial Acetic Acid | 70 |
| Water | 6 |
| Phosphine Oxide | 4-8 |

Two phosphine oxides were tested in this example.

The first was TPPO, which served as a comparative example.

The second was an embodiment of the at least one tri-aliphatic hydrocarbyl phosphine oxide provided herein. Specifically, the embodiment of the at least one tri-aliphatic hydrocarbyl phosphine oxide of this example was a combination of the following four tri-aliphatic hydrocarbyl phosphine oxides: (CH₃(CH₂)₅)₃PO, (CH₃(CH₂)₇)₃PO, (CH₃(CH₂)₅)₂(CH₃(CH₂)₇)₁PO, and (CH₃(CH₂)₅)₁(CH₃(CH₂)₇)₂PO. The embodiment of the at least one tri-aliphatic hydrocarbyl phosphine oxide used in this example is referred to herein as “4HOPO”.

The GAA liquids of Table 1 mimic the compositions or reactor liquids produced during a process for the production of GAA. A composition or reactor liquid can include a number of other species that may be present at an amount of less than 1%, by weight, based on the weight of the composition or reactor liquid. Table 1 also does not include TPPO decomposition products, TDP (i.e., TPPO decompositions products, most of which are unknown, P-containing compounds), and other impurities, since knowledge of their identities is imperfect. Methyl iodide (Mel) and hydrogen iodide (HI), which may be present in compositions or reactor liquids at amount of about 13% and 5%, by weight, were omitted from the study of this example, due to concerns regarding exposure. These omissions were not believed to impact undesirably, if at all, the results of this example or the usefulness of the tests.

The apparatus used for the surface tension measurements of this example was sensitive to drafts, so it could not be used in a fume hood. Therefore, a snorkel was used over the apparatus. The snorkel did not disturb the measurements, and controlled acetic acid vapors; an acetic acid detector was used to confirm this conclusion.

The experiments of this example were used to estimate the effect of water concentration, TPPO concentration, and 4HOPO concentration on the surface tension of the GAA liquids. A three factor, two level, full factorial design was used. The design included a center point. Formally, the design was run with full duplicates, but each replicate was actually an average of three separate determinations. This approach minimized the effect of experimental error on the variance when the data is analyzed. The factor values used are presented at the following table.

TABLE 2
High and Low Factor Values

| | Weight Percent | | M | |
|---|---|---|---|---|
| Factor | Low Value | High Value | Low Value | High Value |
| Water | 2 | 8 | 1.32 | 5.29 |
| TPPO | 0 | 8 | 0.00 | 0.340 |
| 4HOPO | 0 | 8 | 0.00 | 0.280 |

The liquids were prepared in 40 mL scintillation vials. Each liquid was prepared by weighing the appropriate amount of acetic acid, water, TPPO, and 4HOPO prescribed to a total liquid mass of 40 g.

The liquid was then transferred to a crystallization dish (60 mm×35 mm), which had been thoroughly washed, rinsed, and blown dry with nitrogen to ensure it was free from contamination. A calibrated DUNOUY® 70545 tensiometer (CSC Scientific Company, Inc., USA) was utilized for the surface tension measurements. Its platinum-iridium ring, of known size and surface area, was heated to a dull red in the flame of a Bunsen burner to ensure it was free from contamination, prior to affixing it to the instrument. Measurements of each liquid were made in triplicate; the crystallization dish and platinum-iridium ring were cleaned in between the measurements.

The following table describes the 18 experiments of this example, generated with MINITAB®, with the results of the individual measurements and sample average. When analyzing the results, molarity was used rather than weight percentage; Table 2 can be used to translate the Factor Values given in Table 3 from weight percentage to M.

TABLE 3
Surface Tension Measurements

| Design of Experiments | | | | Factor Values | | | Surface Tension Measurements | | | |
|---|---|---|---|---|---|---|---|---|---|---|
| STD | RUN | CTR | | (Weight Percent) | | | (mN/m) | | | |
| Order | Order | Point | Blocks | TPPO | 4HOPO | H₂O | Run 1 | Run 2 | Run 3 | Average |
| 1 | 1 | 1 | 1 | 0 | 0 | 2 | 28 | 26 | 29 | 27.67 |
| 2 | 2 | 1 | 1 | 8 | 0 | 2 | 32 | 33 | 31.5 | 32.17 |
| 3 | 3 | 1 | 1 | 0 | 8 | 2 | 32.5 | 32 | 30.5 | 31.67 |
| 4 | 4 | 1 | 1 | 8 | 8 | 2 | 31.5 | 31 | 30.5 | 31.00 |
| 5 | 5 | 1 | 1 | 0 | 0 | 8 | 31.5 | 32 | 33 | 32.17 |
| 6 | 6 | 1 | 1 | 8 | 0 | 8 | 34.5 | 34 | 33.5 | 34.00 |
| 8 | 7 | 1 | 1 | 0 | 8 | 8 | 32.5 | 33 | 33 | 32.83 |
| 7 | 8 | 1 | 1 | 8 | 8 | 8 | 33 | 32 | 32.5 | 32.50 |
| 9 | 9 | 0 | 1 | 4 | 4 | 5 | 33 | 32 | 32 | 32.33 |
| 10 | 10 | 1 | 2 | 0 | 0 | 2 | 27 | 27 | 28 | 27.33 |
| 11 | 11 | 1 | 2 | 8 | 0 | 2 | 31.5 | 32 | 31 | 31.50 |
| 12 | 12 | 1 | 2 | 0 | 8 | 2 | 31 | 31 | 32 | 31.33 |
| 13 | 13 | 1 | 2 | 8 | 8 | 2 | 30 | 31 | 31 | 30.67 |
| 14 | 14 | 1 | 2 | 0 | 0 | 8 | 31.5 | 32 | 34 | 32.50 |
| 15 | 15 | 1 | 2 | 8 | 0 | 8 | 34 | 35 | 32.5 | 33.83 |
| 16 | 16 | 1 | 2 | 0 | 8 | 8 | 32 | 33.5 | 33.5 | 33.00 |
| 17 | 17 | 1 | 2 | 8 | 8 | 8 | 33 | 32.5 | 34 | 33.17 |
| 18 | 18 | 0 | 2 | 4 | 4 | 5 | 33.5 | 33 | 32.5 | 33.00 |

The data of Table 3 were analyzed utilizing MINITAB®, with concentrations transformed to molarity. A Box-Cox transformation (λ=0; natural log) of the system gave a slightly better fit than did a linear fit (Table 4A-4C).

TABLE 4A
Box-Cox Transformation

| | | Adj | Adj | F- | P- |
|---|---|---|---|---|---|
| Source | DF | SS | MS | Value | Value |
| Model | 7 | 0.057197 | 0.008171 | 30.67 | 0.000 |
| Linear | 3 | 0.036788 | 0.012263 | 46.03 | 0.000 |
| H₂O (M) | 1 | 0.027567 | 0.027567 | 103.49 | 0.000 |
| TPPO (M) | 1 | 0.007227 | 0.007227 | 27.13 | 0.000 |
| 4HOPO (M) | 1 | 0.001994 | 0.001994 | 7.48 | 0.021 |
| 2-Way Interactions | 3 | 0.016971 | 0.005657 | 21.24 | 0.000 |
| H₂O | 1 | 1.001586 | 0.001586 | 5.95 | 0.035 |
| (M) *TPPO (M) | | | | | |
| H₂O | 1 | 0.003507 | 0.003507 | 13.17 | 0.005 |
| (M) *4HOPO (M) | | | | | |
| TPPO (M) *4HOPO (M) | 1 | 0.011878 | 0.011878 | 44.59 | 0.000 |
| 3-Way Interactions | 1 | 0.003438 | 0.003438 | 12.91 | 0.005 |
| H₂O | 1 | 0.003438 | 0.003438 | 12.91 | 0.005 |
| (M) *TPPO | | | | | |
| (M) *4HOPO (M) | | | | | |
| Error | 10 | 0.002664 | 0.000266 | | |
| Curvature | 1 | 0.001762 | 0.001762 | 17.59 | 0.002 |
| Pure Error | 9 | 0.000902 | 0.000100 | | |
| Total | 17 | 0.059861 | | | |

TABLE 4B
Model Summary for Transformed Response

| S | R-sq | R-sq (adj) | R-sq (pred) |
|---|---|---|---|
| 0.0163213 | 95.55% | 92.43% | 90.90% |

TABLE 4C
Coded Coefficients for Transformed Response

| Term | Effect | Coef | SE Coef | T-Value | P-Value | VIF |
|---|---|---|---|---|---|---|
| Constant | | 3.45832 | 0.00385 | 898.97 | 0.000 | |
| H₂O (M) | 0.08302 | 0.04251 | 0.00408 | 10.17 | 0.000 | 1.00 |
| TPPO (M) | 0.04251 | 0.02125 | 0.00408 | 5.21 | 0.000 | 1.00 |
| 4HOPO (M) | 0.02232 | 0.01116 | 0.00408 | 2.74 | 0.021 | 1.00 |
| H₂O (M) *TPPO (M) | −0.01991 | −0.00996 | 0.00408 | −2.44 | 0.035 | 1.00 |
| H₂O (M) *4HOPO (M) | −0.02961 | −0.01480 | 0.00408 | −3.63 | 0.005 | 1.00 |
| TPPO (M) *4HOPO (M) | −0.05449 | −0.02725 | 0.00408 | −6.68 | 0.000 | 1.00 |
| H₂O (M) *TPPO (M) | 0.02932 | 0.01466 | 0.00408 | 3.59 | 0.005 | 1.00 |
| *4HOPO (M) | | | | | | |

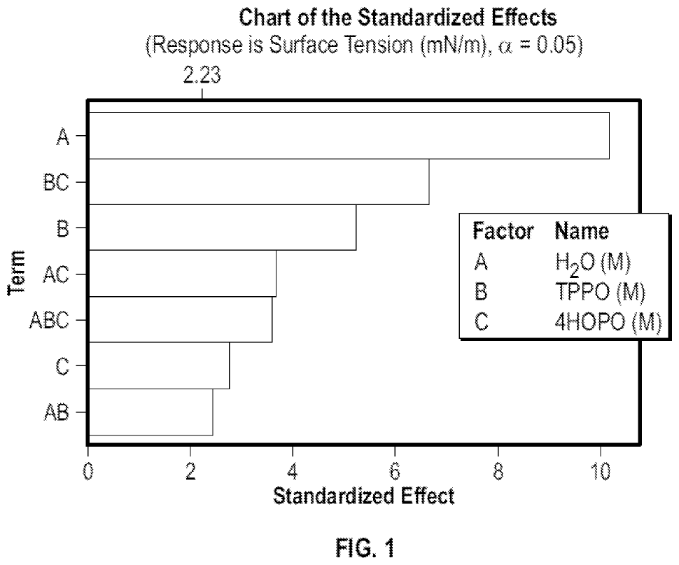

All effects and interactions were found to be significant in predicting surface tension with >95% confidence (FIG. 1). FIG. 1 is a Pareto chart, which demonstrates that all main effects and interactions were significant at >95% confidence. The regression equation was determined to be—

ln(ST (mN/m))=3.26+0.0408 H₂O (M)+0.524 TPPO (M)+0.636 4HOPO (M)−0.0726 H2O (M)*TPPO (M)−0.108 H2O (M)*4HOPO (M)−2.20 TPPO (M)*4HOPO (M)+0.314 H2O (M)*TPPO (M)*4HOPO (M)

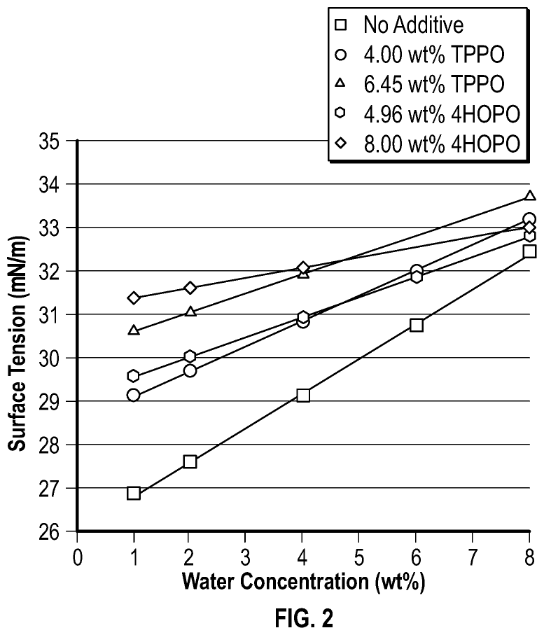

In systems with significant interaction, main effects can be confounded with the higher level interactions. Therefore, the easiest method to estimate changes in response due to factor variation can include utilizing the regression equation as a model to estimate the response with factor values of interest. Table 5 shows cases that were used to analyze the effects of variations in the water, TPPO, and 4HOPO. The cases with 4 wt % TPPO were chosen. The cases with 4.96 wt % 4HOPO were chosen because this concentration is equimolar with the 4 wt % TPPO concentration. The predicted surface tension for each of case is given at Table 6, and is plotted at FIG. 2 vs. water concentration.

TABLE 5
Cases for Analyzing the Effects of Water, TPPO,
and 4HOPO Variation on GAA Liquid Surface Tension

| | | | | | | |
|---|---|---|---|---|---|---|
| Water | TPPO = | 4HOPO = | TPPO = | TPPO = | 4HOPO = | 4HOPO = |
| (wt %) | 0 wt % | 0 wt % | 4.00 wt % | 6.45 wt % | 4.96 wt % | 8.00 wt % |

| | | | | | |
|---|---|---|---|---|---|
| 1 | Case N-1 | Case T1-1 | Case T2-1 | Case L1-1 | Case L2-1 |
| 2 | Case N-2 | Case T1-2 | Case T2-2 | Case L1-2 | Case L2-2 |
| 4 | Case N-3 | Case T1-3 | Case T2-3 | Case L1-3 | Case L2-3 |
| 6 | Case N-4 | Case T1-4 | Case T2-4 | Case L1-4 | Case L2-4 |
| 8 | Case N-5 | Case T1-5 | Case T2-5 | Case L1-5 | Case L2-5 |

TABLE 6
Predicted Surface Tension of Liquids of Table 5

| | (wt %) | (M) |
|---|---|---|
| Case N-1 | | |
| H₂0 | 8.00 | 5.29 |
| TPPO | 0.00 | 0.000 |
| 4HOPO | 0 | 0.000 |
| ln(ST) | 3.48 | |
| ST mN/m | 32.4 | |
| Case N-2 | | |
| H₂0 | 6.00 | 3.97 |
| TPPO | 0.00 | 0.000 |
| 4HOPO | 0 | 0.000 |
| ln(ST) | 3.43 | |
| ST mN/m | 30.7 | |
| Case N-3 | | |
| H₂0 | 4.00 | |
| TPPO | 0.00 | |
| 4HOPO | 0 | |
| ln(ST) | 3.37 | |
| ST mN/m | 29.1 | |
| Case N-4 | | |
| H₂0 | 2.00 | 1.32 |
| TPPO | 0.00 | 0.000 |
| 4HOPO | 0 | 0.000 |
| ln(ST) | 3.32 | |
| ST mN/m | 27.6 | |
| Case N-5 | | |
| H₂0 | 1.00 | 0.66 |
| TPPO | 0.00 | 0.000 |
| 4HOPO | 0 | 0.000 |
| ln(ST) | 3.29 | |
| ST mN/m | 26.9 | |
| Case T1-1 | | |
| H₂0 | 8.00 | 5.29 |
| TPPO | 4.00 | 0.171 |
| 4HOPO | 0 | 0.000 |
| ln(ST) | 3.50 | |
| ST mN/m | 33.2 | |
| Case T1-2 | | |
| H₂0 | 6.00 | 3.97 |
| TPPO | 4.00 | 0.171 |
| 4HOPO | 0 | 0.000 |
| ln(ST) | 3.47 | |
| ST mN/m | 32.0 | |
| Case T1-3 | | |
| H₂0 | 4.00 | 2.64 |
| TPPO | 4.00 | 0.171 |
| 4HOPO | 0 | 0.000 |
| ln(ST) | 3.43 | |
| ST mN/m | 30.8 | |
| Case T1-4 | | |
| H₂0 | 2.00 | |
| TPPO | 4.00 | |
| 4HOPO | 0 | |
| ln(ST) | 3.39 | |
| ST mN/m | 29.7 | |
| Case T1-5 | | |
| H₂0 | 1.00 | 0.66 |
| TPPO | 4.00 | 0.171 |
| 4HOPO | 0 | 0.000 |
| ln(ST) | 3.37 | |
| ST mN/m | 29.1 | |
| Case L1-1 | | |
| H₂0 | 8.00 | 5.29 |
| TPPO | 0.00 | 0.000 |
| 4H0P0 | 4.96 | 0.171 |
| ln(ST) | 3.49 | |
| ST mN/m | 32.8 | |
| Case L1-2 | | |
| H₂0 | 6.00 | 3.97 |
| TPPO | 0.00 | 0.000 |
| 4HOPO | 4.96 | 0.171 |
| ln(ST) | 3.46 | |
| ST mN/m | 31.9 | |
| Case L1-3 | | |
| H₂0 | 4.00 | 2.64 |
| TPPO | 0.00 | 0.000 |
| 4HOPO | 4.96 | 0.171 |
| ln(ST) | 3.43 | |
| ST mN/m | 30.9 | |
| Case L1-4 | | |
| H₂0 | 2.00 | 1.32 |
| TPPO | 0.00 | 0.000 |
| 4HOPO | 4.96 | 0.171 |
| ln(ST) | 3.40 | |
| ST mN/m | 30.0 | |
| Case L1-5 | | |
| H₂0 | 1.00 | 0.66 |
| TPPO | 0.00 | 0.000 |
| 4HOPO | 4.96 | 0.171 |
| ln(ST) | 3.39 | |
| ST mN/m | 29.6 | |
| Case T2-1 | | |
| H₂0 | 8.00 | 5.29 |
| TPPO | 6.45 | 0.276 |
| 4HOPO | 0 | 0.000 |
| ln(ST) | 3.52 | |
| ST mN/m | 33.7 | |
| Case T2-2 | | |
| H₂0 | 6.00 | 3.97 |
| TPPO | 6.45 | 0.276 |
| 4HOPO | 0 | 0.000 |
| ln(ST) | 3.49 | |
| ST mN/m | 32.8 | |
| Case T2-3 | | |
| H₂0 | 4.00 | 2.64 |
| TPPO | 6.45 | 0.276 |
| 4HOPO | 0 | 0.000 |
| ln(ST) | 3.46 | |
| ST mN/m | 31.9 | |
| Case T2-4 | | |
| H₂0 | 2.00 | 1.32 |
| TPPO | 6.45 | 0.276 |
| 4HOPO | 0 | 0.000 |
| ln(ST) | 3.44 | |
| ST mN/m | 31.1 | |
| Case T2-5 | | |
| H₂0 | 1.00 | 0.66 |
| TPPO | 6.45 | 0.276 |
| 4HOPO | 0 | 0.000 |
| ln(ST) | 3.42 | |
| ST mN/m | 30.6 | |
| Case T2-3 | | |
| H₂0 | 4.00 | 2.64 |
| TPPO | 6.45 | 0.276 |
| 4HOPO | 0 | 0.000 |
| ln(ST) | 3.46 | |
| ST mN/m | 31.9 | |
| Case T2-4 | | |
| H₂0 | 2.00 | 1.32 |
| TPPO | 6.45 | 0.276 |
| 4HOPO | 0 | 0.000 |
| ln(ST) | 3.44 | |
| ST mN/m | 31.1 | |
| Case T2-5 | | |
| H₂0 | 1.00 | 0.66 |
| TPPO | 6.45 | 0.276 |
| 4HOPO | 0 | 0.000 |
| ln(ST) | 3.42 | |
| ST mN/m | 30.6 | |
| Case L2-1 | | |
| H₂0 | 8.00 | 5.29 |
| TPPO | 0.00 | 0.000 |
| 4HOPO | 8 | 0.275 |
| ln(ST) | 3.50 | |
| ST mN/m | 33.0 | |
| Case L2-1 | | |
| H₂0 | 8.00 | 5.29 |
| TPPO | 0.00 | 0.000 |
| 4HOPO | 8 | 0.275 |
| ln(ST) | 3.50 | |
| ST mN/m | 33.0 | |
| Case L2-2 | | |
| H₂0 | 6.00 | 3.97 |
| TPPO | 0.00 | 0.000 |
| 4HOPO | 8 | 0.275 |
| ln(ST) | 3.48 | |
| ST mN/m | 32.6 | |
| Case L2-3 | | |
| H₂0 | 4.00 | 2.64 |
| TPPO | 0.00 | 0.000 |
| 4HOPO | 8 | 0.275 |
| ln(ST) | 3.47 | |
| ST mN/m | 32.1 | |
| Case L2-4 | | |
| H₂0 | 2.00 | 1.32 |
| TPPO | 0.00 | 0.000 |
| 4HOPO | 8 | 0.275 |
| ln(ST) | 3.45 | |
| ST mN/m | 31.6 | |
| Case L2-5 | | |
| H₂0 | 1.00 | 0.66 |
| TPPO | 0.00 | 0.000 |
| 4HOPO | 8 | 0.275 |
| ln(ST) | 3.45 | |
| ST mN/m | 31.4 | |

Water, as predicted from the Pareto chart at FIG. 1, had a strong effect on surface tension. As water was reduced from 8 wt % to 1 wt % when no additive (i.e., TPPO or 4HOPO) was present, the model predicted that surface tension would decrease by 17%.

The behavior of the system as the additive identity was varied was not as simply predicted from the Pareto chart as it was for water variation. In the range between 6 wt % water and 4 wt % water, the identity of the additive had little effect on surface tension. Above or below that range, divergence was noted. 4HOPO was more effective at low water in maintaining surface tension as water was decreased. Surpassingly, below 4 wt % water, 4HOPO increasingly was found to be more effective than TPPO at mitigating the decrease in surface tension occasioned by decreasing water concentration. If water was increased above that 4 wt % to 6 wt % range, then the TPPO solutions had higher surface tensions than the 4HOPO solutions.

Increasing additive concentration as water was lowered was an effective means of maintaining surface tension with both TPPO and 4HOPO. With each additive, when water was reduced from 6 wt % to 4 wt % (Case T1-2->Case T1-3 and Case L1-2 to Case L1-3) surface tension was reduced. However, increasing additive concentration by 60% (Case T2-3 and Case L2-3) restored the surface tension to the values observed for Case T1-2 and Case L1-2. The ability to tune surface tension by additive concentration and additive identity changes suggested that any increased entrainment at low water due to decreases GAA reactor solution surface tension could be mitigated utilizing these factors.

Other species in a GAA reactor solution may impact surface tension, but, as Table 6 shows, useful at least semi-quantitative predictions can be made.

What is claimed is:

1. A composition comprising:
acetic acid;
water; and
at least one tri-aliphatic hydrocarbyl phosphine oxide;
wherein the acetic acid is present in the composition at the amount of 60% to about 80%, by weight, based on the weight of the composition;
wherein the water is present in the composition at the amount of 0.1% to about 6%, by weight, based on the weight of the composition; and
wherein the at least one tri-aliphatic hydrocarbyl phosphine oxide is present in the composition at the amount of 2% to about 20%, by weight, based on the weight of the composition.

2. The composition of claim 1, wherein the at least one tri-aliphatic hydrocarbyl phosphine oxide is a compound of formula (I): 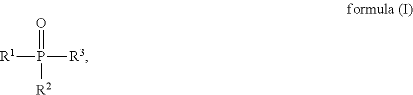
wherein R¹, R², and R³ are independently selected from a monovalent C₁-C₂₀ aliphatic hydrocarbyl.

3. The composition of claim 2, wherein R¹, R², and R³ are independently selected from a monovalent C₄-C₁₅ aliphatic hydrocarbyl.

4. The composition of claim 2, wherein R¹, R², and R³ are independently selected from a monovalent C₆-C₈ aliphatic hydrocarbyl.

5. The composition of claim 2, wherein—
(i) R¹, R², and R³ are n-octyl groups,
(ii) R¹, R², and R³ are n-hexyl groups,
(iii) R¹ and R² are n-hexyl groups, and R³ is an n-octyl group, or
(iv) R¹ and R² are n-octyl groups, and R³ is an n-hexyl group.

6. The composition of claim 1, wherein the at least one tri-aliphatic hydrocarbyl phosphine oxide is a compound of formula (Ia), a compound of formula (Ib), a compound of formula (Ic), and a compound of formula (Id): 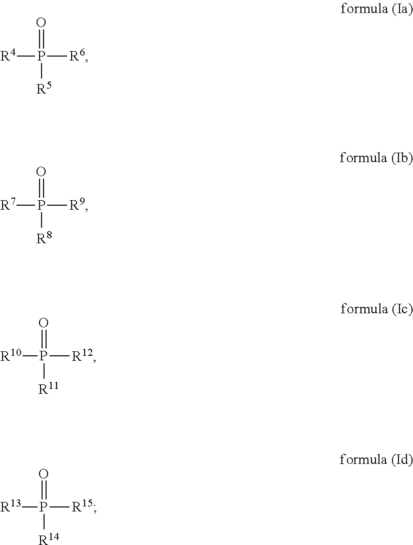
wherein—
(i) each of R⁴, R⁵, and R⁶ is a monovalent aliphatic C₅-C₇ hydrocarbyl,
(ii) each of R⁷, R⁸, and R⁹ is a monovalent aliphatic C₇-C₉ hydrocarbyl,
(iii) R¹⁰, R¹¹, and R¹² are independently selected from a monovalent aliphatic C₅-C₉ hydrocarbyl, and
(iv) R¹³, R¹⁴, and R¹⁵ are independently selected from a monovalent aliphatic C₅-C₉ hydrocarbyl.

7. The composition of claim 6, wherein—
(i) each of R⁴, R⁵, and R⁶ is an n-hexyl group,
(ii) each of R⁷, R⁸, and R⁹ is an n-octyl group,
(iii) R¹⁰ and R¹¹ are n-hexyl groups, and R¹² is an n-octyl group, and
(iv) R¹³ and R¹⁴ are n-octyl groups, and R¹¹ is an n-hexyl group.

8. The composition of claim 1, wherein the at least one tri-aliphatic hydrocarbyl phosphine oxide is present in the composition at the amount of 3% to about 10%, by weight, based on the weight of the composition.

9. A method for the production of acetic acid, the method comprising:
contacting (i) methanol, (ii) carbon monoxide, (iii) water, (iv) a carbonylation catalyst, and (iv) at least one tri-aliphatic hydrocarbyl phosphine oxide in a carbonylation reactor to produce a reactor liquid, wherein the reactor liquid comprises the acetic acid, and
wherein the acetic acid is present in the reactor liquid at the amount of 50% to 80%, by weight, based on the weight of the reactor liquid; wherein the water is present in the reactor liquid at the amount of 0.1% to 6%, by weight, based on the weight of the reactor liquid; and wherein the at least one tri-aliphatic hydrocarbyl phosphine oxide is present in the reactor liquid at the amount of 2% to 20%, by weight, based on the weight of the reactor liquid.

10. The method of claim 9, wherein the acetic acid is present in the reactor liquid at the amount of 60% to 80%, by weight, based on the weight of the reactor liquid; wherein the water is present in the reactor liquid at the amount of 1.0% to 6%, by weight, based on the weight of the reactor liquid; and wherein the at least one tri-aliphatic hydrocarbyl phosphine oxide is present in the reactor liquid at the amount of 2% to 18%, by weight, based on the weight of the reactor liquid.

11. The method of claim 10, wherein the at least one tri-aliphatic hydrocarbyl phosphine oxide is present in the reactor liquid at the amount of 3% to about 10%, by weight, based on the weight of the reactor liquid.

12. The method of claim 9, wherein the at least one tri-aliphatic hydrocarbyl phosphine oxide is a compound of formula (I): 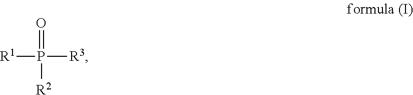
wherein R¹, R², and R³ are independently selected from a monovalent C₁-C₂₀ aliphatic hydrocarbyl.

13. The method of claim 12, wherein R¹, R², and R³ are independently selected from a monovalent C₄-C₁₅ aliphatic hydrocarbyl.

14. The method of claim 12, wherein R¹, R², and R³ are independently selected from a monovalent C₆-C₈ aliphatic hydrocarbyl.

15. The method of claim 12, wherein—
(i) R¹, R², and R³ are n-octyl groups,
(ii) R¹, R², and R³ are n-hexyl groups,
(iii) R¹ and R² are n-hexyl groups, and R³ is an n-octyl group, or
(iv) R¹ and R² are n-octyl groups, and R³ is an n-hexyl group.

16. The method of claim 9, wherein the at least one tri-aliphatic hydrocarbyl phosphine oxide is a compound of formula (Ia), a compound of formula (Ib), a compound of formula (Ic), and a compound of formula (Id): 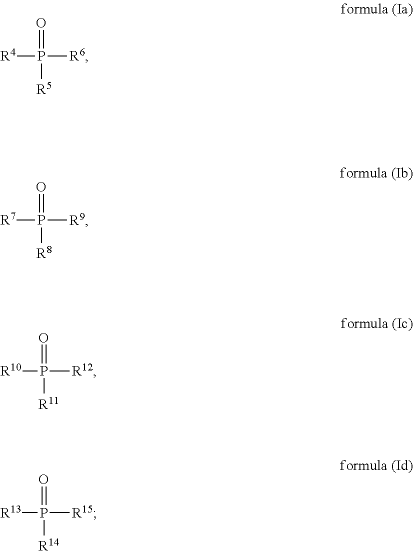
wherein—
(i) each of R⁴, R⁵, and R⁶ is a monovalent aliphatic C₅-C₇ hydrocarbyl,
(ii) each of R⁷, R⁸, and R⁹ is a monovalent aliphatic C₇-C₉ hydrocarbyl,
(iii) R¹⁰, R¹¹, and R¹² are independently selected from a monovalent aliphatic C₅-C₉ hydrocarbyl, and
(iv) R¹³, R¹⁴, and R¹⁵ are independently selected from a monovalent aliphatic C₅-C₉ hydrocarbyl.

17. The method of claim 16, wherein—
(i) each of R⁴, R⁵, and R⁶ is an n-hexyl group,
(ii) each of R⁷, R⁸, and R⁹ is an n-octyl group,
(iii) R¹⁰ and R¹¹ are n-hexyl groups, and R¹² is an n-octyl group, and
(iv) R¹³ and R¹⁴ are n-octyl groups, and R¹⁵ is an n-hexyl group.

18. The method of claim 9, wherein the carbonylation catalyst comprises rhodium.

19. The method of claim 18, wherein the carbonylation catalyst comprises rhodium metal.

20. The method of claim 18, wherein the carbonylation catalyst comprises a rhodium compound selected from the group consisting of Rh₂(CO)₄I₂, Rh₂(CO)₄Br₂, Rh₂(CO)₄C₁₂, Rh(CH₃CO₂)₂, Rh(CH₃CO₂)₃, and [H]Rh(CO)₂I₂.
